(19) United States
(12) Patent Application Publication
(10) Pub. No.: US 2010/0104614 A1
(43) Pub. Date: Apr. 29, 2010

(54) PROVIDONE COMPOSITIONS FOR WOUND HEALING

(76) Inventor: Bryan T. ORONSKY, Los Altos Hills, CA (US)

Correspondence Address: MORRISON & FOERSTER LLP, 755 PAGE MILL RD, PALO ALTO, CA, 94304-1018, US

(21) Appl. No.: 12/493,000

(22) Filed: Jun. 26, 2009

Related U.S. Application Data
(60) Provisional application No. 61/076,474, filed on Jun. 27, 2008.

(51) Int. Cl. A61K 31/79; C08F 26/10; A61K 9/70; A61P 17/02

(52) U.S. Cl. 424/402; 526/264; 424/445; 424/78.32

(57) ABSTRACT

Described here are wound dressings and kits. The wound dressings include povidone compositions that may be directly applied to wound surfaces or provided on substrates to form a composite material. The povidone compositions contain a povidone polymer, where the povidone polymer itself is capable of healing wounds. The povidone compositions may be in particulate form. In other instances, the povidone compositions are formulated as gels, ointments, pastes, films, or other topically applied compositions. Methods for applying the povidone compositions to treat wounds are also described.

### CROSS-REFERENCE TO RELATED APPLICATIONS

This application claims priority to U.S. Provisional Patent Application Ser. No. 61/076,474 filed on Jun. 27, 2008, which is hereby incorporated by reference in its entirety.

### FIELD

Described here are wound dressings and kits. The wound dressings include povidone compositions that may be directly applied to wound surfaces or provided on substrates to form a composite material. Methods for applying the povidone compositions to treat wounds are also described.

### BACKGROUND

The general treatment of wounds begins with a proper assessment. Among other factors, size and depth of the wound is determined, as well as whether foreign bodies or necrotic tissue is present. Given that infection prevents wound healing, a topical anti-infective is usually employed as part of the wound care regimen. Systemic antibiotic therapy may be indicated in cases where necrotic tissue is present.

Povidone-iodine (polyvinylpyrrolidone-iodine complex; PVP-iodine) is a widely used topical anti-infective. It is part of the family of iodophors, which are comprised of readily dissociable, loose complexes of tri-iodide or iodine with polymers or surfactants. Iodophors not only increase the solubility of iodine in aqueous media, but reduce its chemical potential and vapor pressure, thereby reducing its undesirable side effects. The iodophors serve as reservoirs of iodine and function by slowly releasing iodine at the site of application. Povidone-iodine is utilized in commercially available disinfectant products such as Betadine® (Purdue Pharma, Stamford, Conn.) and Isodine® (Meiji Seika KK, Tokyo) topical antiseptics, which are widely used in hospitals for prepping of skin prior to surgery and as surgical scrubs and hand washes for health care personnel.

Although they are useful for application to intact skin, iodophor solutions are not well suited for use on wounds. When applied, the iodine in contact with the wound far exceeds minimum inhibitory concentrations by several orders of magnitude. At these concentrations, the iodine is generally thought to exert toxic effects on the wound tissue as well as on cells, such as fibroblasts, involved in the wound repair process. As a result, the wound repair process is retarded. In the case of chronic wounds (e.g., ulcers), topical anti-infectives alone are generally ineffective.

Accordingly, wound care management employing non-toxic substances would be useful. Wound dressings for promoting faster healing would also be desirable. In particular, wound dressings capable of healing chronic wounds would be desirable.

### SUMMARY

Described here are compositions, methods, and kits for treating wounds. The compositions and kits include povidone. The povidone compositions form wound dressings, and are used to treat various types of wounds, including chronic wounds.

In some variations, the wound dressing includes a povidone composition consisting solely of povidone particles (powder). The particles may be dispersed directly on the wound. In other variations, povidone is formulated into a topical composition such as a solution, gel, ointment, or paste, and then applied to the wound. Alternatively, the powder and other topical dosage forms may be sprinkled or spread on a substrate and then the substrate applied to the wound. In yet other variations, the substrate is impregnated with the povidone composition. Kits may also be made from the povidone compositions described herein, and configured for use with specific wound types. For example, the kits may be configured for treating ischemic ulcers due to diabetes.

### DETAILED DESCRIPTION

Surprisingly, it has been discovered that povidone alone is capable of healing wounds, including chronic wounds. Without being bound by theory, it is hypothesized that the wound healing effect of povidone may be due to in part to the skin penetration properties of povidone. It is also hypothesized that povidone may have antimicrobial and anti-inflammatory properties. In view of this, described herein are wound dressings that include povidone compositions, where the povidone polymer itself is capable of healing the wound. The povidone composition may include additional ingredients, such as additional polymers, penetration enhancers, odor-controlling agents, opacifiers, antioxidants, fragrance, colorants, gelling agents, thickening agents, stabilizers, surfactants, and the like, but the wound healing properties of the composition will be entirely attributable to the povidone polymer. Trace amounts of vitamins, minerals, and/or herbs may also be included, but again, the wound healing properties of the composition will be entirely attributable to the povidone polymer.

The povidone compositions may solely include povidone in powered or particulate form. The powder may be applied, e.g., by shaking onto the wound surface or onto a substrate for application to the wound surface. The povidone compositions may also take other topical forms, such as solutions, gels, ointments, pastes, sprays, etc. These topical forms may also be placed directly on the wound or onto a substrate prior to application on the wound.

The povidone compositions may be used to treat any type of wound. For example, the compositions may be used to treat burns, ulcers (e.g., diabetic ulcers, ischemic ulcers, venous stasis ulcers), pressure sores, surgical wounds and excisions, traumatic wounds, abrasions, and lacerations. Methods for administering the compositions, as well as kits with the compositions and/or substrates contained therein are also described.

### I. COMPOSITIONS

The compositions described here include povidone in various forms. The composition may be in particulate form, such as a powder, or formulated as another type of topical dosage form, e.g., a solution, gel, ointment, paste, or spray, by mixing with water, other polymers and additives, and the like. The wound dressings are formed by placing the povidone composition onto a wound surface directly or by using a substrate. As used herein, the term “dressing” refers to a composition or composite material suitable for application to wounds. The wounds may be of any size, shape, and depth, located anywhere on the body, and affect any type of tissue.

1) Povidone Powder

Any type of povidone may be used in making the compositions described here. In one variation, a crosslinked povidone (e.g., crospovidone) is used. In another variation, a povidone copolymer (e.g., copovidone) is employed. Here the povidone in its particulate form is applied to a wound surface to form the wound dressing. The wound dressing consists essentially of particulate povidone. The povidone particles may be of any diameter and geometry, so long as they function as a free-flowing powder.

Alternatively, the povidone particles may be placed, e.g., by sprinkling, pouring, etc., onto a substrate. The surface of the substrate with the particulate povidone would then be placed on the wound so that the povidone directly contacts the wound surface. The substrate may be configured to self-adhere to the skin around the wound or be secured in other conventional manners, e.g., by taping, ace wrapping, etc. In some variations, the povidone particles are impregnated within the substrate.

2) Other Topical Povidone Compositions

The povidone compositions described herein may be formulated into any topical dosage form. The topical dosage forms may be creams, lotions, solutions, gels, ointments, pastes, sprays, films, etc. The topical dosage forms include povidone as the sole wound healing agent, and are suitable for application to any wound surface. Thus, if another polymer or substance is included in the topical compositions, it will not be for wound healing purposes. The polymer or substance may serve as, without limitation, a carrier, a viscosity enhancing agent, or an adhesive. For example, the substance may be a preservative such as benzalkonium chloride, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben (sodium), phenol, phenylethyl alcohol, phenylmercuric acetate, phenylmercuric nitrate, potassium benzoate, potassium sorbate, propylparaben, propylparaben (sodium), sodium benzoate, sodium dehydroacetate, sorbic acid, and mixtures thereof. Stabilizers and colorants may also be included.

In some variations, the povidone compositions are in the form of an ointment. The ointment base may be an oleaginous base, an emulsifiable base, an emulsion base, or a water-soluble base. The oleaginous ointment base that may be used includes, without limitation, vegetable oils, fats obtained from animals, and semisolid hydrocarbons obtained from petroleum. Suitable emulsifiable ointment bases that may be used, include, for example, hydroxystearin sulfate, anhydrous lanolin, and hydrophilic petrolatum. Exemplary emulsion ointment bases that may be used are water-in-oil (W/O) emulsions or oil-in-water (O/W) emulsions that include, for example, cetyl alcohol, glyceryl monostearate, lanolin, and stearic acid.

In other variations, the povidone compositions are in the form of a cream. The creams may be viscous liquids or semisolid emulsions, either oil-in-water or water-in-oil. The cream bases may be water-washable, and contain an oil phase, an emulsifier, and an aqueous phase. The oil phase, or internal phase, may be generally comprised of petrolatum and a fatty alcohol such as cetyl or stearyl alcohol. The aqueous phase may be formulated to exceed the oil phase in volume, and contain a humectant.

In yet another variation, the povidone compositions are in the form of a gel. The gels may be semisolid, suspension-type systems. Single-phase gels may contain organic macromolecules distributed substantially uniformly throughout the carrier liquid, which may be aqueous, but may also contain an alcohol and, optionally, an oil. Exemplary organic macromolecules that may be used in the gels, include, but are not limited to, carbomers; hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin.

In yet further variations, the povidone compositions are in the form of a lotion. The lotions may be formulated as suspensions of solids and contain suspending agents to produce better dispersions. Examples of such suspending agents include methylcellulose and sodium carboxymethylcellulose.

The povidone compositions may also be formulated pastes. Pastes are semisolid dosage forms in which the active agent is suspended in a suitable base. Depending on the nature of the base, pastes are divided between fatty pastes or those made from a single-phase aqueous gels. The base in a fatty paste is generally petrolatum, hydrophilic petrolatum, or the like. The pastes made from single-phase aqueous gels may generally incorporate carboxymethylcellulose or the like as a base.

In some variations, the povidone compositions form a film on the wound surface. To aid film formation, other film forming agents such as, but not limited to, acrylic acid and its derivatives, polyacrylic and its derivatives such as polybutylmethacrylate and polymethacrylic acid, polymethacrylate, ascorbyl palmitate, carbomer, carnauba wax, cellulose derivatives such as cellulose acetate phthalates, rosca mellose sodium, hydroxyethyl cellulose, hydroxypropyl cellulose, hydroxypropyl methylcellulose, ethyl cellulose and related compounds, hydroxypropyl methylcellulose phthalate, hypromellose phthalate, cetyl alcohol and derivatives, microcystalline wax, poloxamer, polyethylene glycol, polyurethane, polyvinyl acetate, polyvinyl acetate phthalate, polyvinyl alcohol, silicone rubber and derivatives, shellac, triglycerides derivatives, and combinations thereof are used.

The povidone compositions can also include at least one film plasticizer agent that may serve to soften the polymer film formed by the film forming agent so that it is sufficiently flexible to move with area of the body applied without cracking or peeling. Examples of suitable film plasticizer agents include, without limitation, polybutylphthalate, benzyl benzoate, dibutyl sebacate, dimethyl phthalate, dibutyl phthalate, triacetin, glycol and derivatives thereof, benzyl benzoate, glycerin, mineral oil, lanolin alcohols (such as C₁₋₈ alcohols), petroleum and lanolin alcohols, polyethylene glycol, glycerin, sorbitol, triacetin, triethylene citrate, propylene glycol, chlorbutanol, castor oil and gelatin.

In some variations, the povidone compositions may be cast into a film prior to application to the wound or applied to the wound directly where they polymerize in situ. A “spread-on” film polymerizes when applied to the skin and may be delivered as a cream or ointment from a tube, roll-on, spray, and the like. The film may be created by incorporating a silicone rubber, such as an addition cure (similar to a gel, but reinforced with silica) or a condensation cure, for example, Dow Corning® 7-5300 FILM-IN-PLACE COATING available from Dow Corning Corporation (Midland, Mich.), into the external phase. Upon mixing with the internal phase, the resultant emulsion is allowed to cure and provides a “spread-on” film, which polymerizes when applied to the wound. The emulsion may be spread onto a substrate to achieve a desired thickness.

In other instances, the povidone compositions may be preformed into a layer or patch. The patch may be of varying thickness. The patch may also be cut to have a shape that generally follows the wound edges.

In some variations, the patches may include a pharmaceutically acceptable adhesive material that serves to affix the patch to the wound or skin. For example, the adhesive material may be a pressure-sensitive adhesive (PSA) including, but not limited to, polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). The adhesive material may mixed into the povidone composition, or the povidone composition layer and adhesive may be provided as separate and distinct layers. Alternatively, the patch may be configured to include povidone compositions in one or more reservoirs in an adhesive layer.

A patch backing layer may also be included, e.g., to provide structural support to the patch, and in certain variations, occlusivity. The backing may be comprised of a flexible elastomeric material that serves as a protective covering to prevent transmission of substances through the upper surface of the patch, and may impart a degree of occlusivity to the patch, such that the area of the body surface covered by the patch becomes hydrated during use. The material used for the backing layer may permit the patch to follow the contours of the wound or skin and be worn comfortably on areas such as joints or other points of flexure that are normally subjected to mechanical strain, with little or no likelihood of the patch disengaging from the wound/skin due to differences in the flexibility or resiliency of the wound/skin and the patch. The materials used as the backing layer may be either occlusive or permeable, as noted above, and may be made from synthetic polymers (e.g., polyester, polyethylene, polypropylene, polyurethane, polyvinyl chloride, and polyether amide), natural polymers (e.g., cellulosic materials), or macroporous woven and nonwoven materials.

During storage and prior to use, the patch may include a release liner. Immediately prior to use, this layer is typically removed so that the patch may be affixed to the wound/skin. The release liner may be prepared as a disposable element that serves only to protect the patch prior to application. The release liner may be formed from a material impermeable to the povidone polymer and other substances contained in the patch, and which is easily stripped from the patch prior to use.

The patches may be fabricated using conventional coating and laminating techniques known in the art. For example, adhesive matrix systems can be prepared by casting a fluid admixture of adhesive, active agent, and carrier onto the backing layer, followed by lamination of the release liner. Similarly, the adhesive mixture may be cast onto the release liner, followed by lamination of the backing layer. The patches may also be fabricated by solvent evaporation, film casting, melt extrusion, thin film lamination, die cutting, or the like.

In certain variations, an adhesive overlayer that also serves as a patch backing is used to better secure the patch to the body surface. This overlayer is sized such that it extends beyond the patch edges so that adhesive on the overlayer comes into contact with the body surface. The overlayer is useful because the adhesive may lose its adhesion a few hours after application due to hydration. By incorporating such an adhesive overlayer, the patch remains in place for the required period of time.

When used as a spray, the povidone compositions may include at least one organic solvent, e.g., a volatile organic solvent. Volatile solvents that may be employed in this instance, include, but are not limited to, alcohols, for example C₁₋₁₀ alcohols, such as benzyl alcohol, ethanol, methanol, butanol, isobutanol, diacetone alcohol; chlorinated hydrocarbons such as methylene chloride, carbon tetrachloride, trichloroethylene, chlorothene SM; esters such as methyl acetate, ethyl acetate, n-propyl acetate, n-butyl acetate, isobutyl acetate, amyl acetate, 2-thyl hexyl acetate, DuPont DBE, Exxate 500, 700, 900; glycol and ether/ester derivatives, ethylene glycol, PM acetate, butyl celluosolve, carbritol acetate, butyl carbritol acetate, Ektapro EEP; hydrocarbons such as toluene, eyxlene, VM & P naphtha, mineral spirits, Aromatic 100, Aromatic 150, ketones such as acetone, methyl ethyl ketone, methyl butyl ketone; ethers such as dimethyl ether; benzyl benzoate; isoptopyl myristate; acetonitrile; ethyl oleate; glycerol, glycofurol (tetraglycol); propylene glycol, polyethylene glycol (PEG); bexane; n-hexane, glycol ethers; methylene chloride; methyl chloride; octyl dodecanol; toluene; trichlorethane; diethyl phthalate; and dibutyl phthalate. Non-volatile solvents may also be used.

Other agents may also be added to prevent spoilage upon storage of the compositions, i.e., to inhibit growth of microbes such as yeasts and molds. Suitable agents are typically selected from the group consisting of the methyl and propyl esters of p-hydroxybenzoic acid (i.e., methyl and propyl paraben), sodium benzoate, sorbic acid, imidurea, and combinations thereof.

The dosage forms may also contain irritation-mitigating additives to minimize or eliminate the possibility of skin irritation. Suitable irritation-mitigating additives include, for example, alpha.-tocopherol; monoamine oxidase inhibitors, e.g., phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine.

3) Substrates

Both the particulate and non-particulate povidone compositions may be directly placed on a wound, or placed on a substrate for application on a wound. Any substrate (carrier) may be used with the povidone compositions described here. For example, woven, non-woven, knitted, foam, and adhesive substrates may be used. Absorbent or non-absorbent substrates may also be used. In some variations, the compositions are sprinkled or spread on the substrate. In other variations, the compositions are impregnated within the substrate. The povidone compositions will generally be adapted to have a viscosity that allows it to be impregnated within a solid substrate.

The povidone compositions may be applied to the substrates in any pattern or configuration. For example, the povidone compositions may cover the entire substrate or only portions thereof. The coating may be applied to the substrate in either a regular pattern or a random pattern. For example, the coating may be configured in either a regular or random pattern of elements such as straight lines, angled lines, curved lines, intersecting lines, dots, circles and geometric shapes, or using a combination of these elements. The coating may be applied to the substrate using well known techniques such as printing, spraying, reverse roll or knife-over-roll coating, or extrusion coating techniques.

In addition to the substrate, the wound dressings may also include an outer support, such as a backing. The support may be integral with or separate from the backing. Backing materials useful in the wounds dressings described here include monolithic films, apertured films, foams, woven fabrics, nonwoven fabrics and composites thereof. In the case of films, the backing may be composed of any of the polymers known to be useful as backing materials. Such polymers include plasticized PVC, polyolefins such as polyethylene or polypropylene, and polyurethane.

The povidone compositions may also be used to coat devices such as needles, lancets, catheters (e.g., intravenous catheters, arterial catheters, PICC lines, central lines, TPN catheters, urinary catheters), feeding tubes, nasogastric tubes, etc., to aid the wound healing process. The coatings may be useful in individuals with poor circulation, diabetes, or immunodeficiency. Here the coatings may also function to prevent infection and/or inflammation. The devices may be pre-coated with the povidone composition: Alternatively, the povidone composition may be provided on or impregnated within a wipe, tissue, pad, and the like, for application to a device surface.

### II. METHODS

Administration

The wound dressings described here may be applied in any manner. When provided in particulate form, the povidone composition may be dispensed directly on the wound. The particles may absorb wound fluid upon contact with the wound to thereafter provide a dressing with a gel or paste-like consistency. In this instance, the particles may be sprinkled to uniformly or non-uniformly coat the wound. The particles may also be sprinkled on a substrate prior to application of the substrate on the wound. Other povidone compositions may be applied directly onto the wound surface or spread on a substrate prior to wound application. In one variation, the particulate povidone is mixed with water, e.g., sterile water, normal saline, or other liquid, and the mixture applied to the wound or onto a wound via a substrate. When the substrate is used to pack a wound, the substrate may be dampened or soaked in a liquid/povidone mixture or solution and then used as packing. For example, a Kerlix™ roll could be wet with a povidone solution and then packed into an ulcer, pressure sore, or surgical dehiscence.

The various povidone compositions may be used in conjunction with other wound care measures such as surgical debridement and irrigation with sterile saline solution. The wound dressings may be applied for any suitable time period. For example, they may be applied over a time period of one day, over several days, over several weeks, or for several months or more. In general, the wound dressings will be reapplied until the wound is healed. The duration of wound treatment with the dressings described here may depend on such factors as the type of wound being treated, wound location, and form of the povidone composition being applied. Depending on the form used, the povidone composition may be removed with water, or wiped or peeled off the wound.

The povidone compositions may be applied to wounds in any amount, in measured doses or less precisely. The compositions may be directly applied to the wound and allowed to remain in place uncovered, or if desired, covered with a substrate. Alternatively, the composition may be furnished to the wound on a substrate bearing the composition. The compositions may be reapplied as necessary or desired. Here again, the amount of the povidone composition employed may depend on such factors as the type of wound being treated, wound location, and form of the povidone composition being applied.

Any amount of the povidone polymer may also be employed in the povidone compositions. For example, the composition may include only a povidone polymer (100% povidone polymer), or the povidone polymer in a range between about 50% to about 90%, between about 50% to about 80%, between about 50% to about 70%, or between about 50% to about 60%, by weight of the povidone composition.

The povidone compositions may also be used in combination with energy-delivering devices. For example, electrical energy, ultrasound, or laser may be employed after the povidone composition is applied to the wound. The energy may be supplied by hand-held or other miniaturized units, TENS units, or fibers. The energy delivery devices may directly contact the wound surface or substrate, if used, or deliver energy from a non-contact distance.

The energy may be delivered for any suitable time period. It may be delivered from one second to about one minute, from about one minute to about three minutes, or from about three minutes to about five minutes or more. The energy may be continuously delivered or delivered in a pulsatile fashion. Frequency, power, and wavelength of the applied energy may also be varied as appropriate, and depend on such factors as the type of energy being used, type of wound being treated, and whether the energy delivery device contacts the wound or substrate.

Wound Types

The povidone compositions described here may be used to treat wounds resulting from any etiology. For example, the wounds may be due to burns, infections, ischemia, lymphedema, neoplasms, neuropathy, radiation damage, surgical procedures, venous insufficiency, and trauma. Risk factors associated with non-healing wounds include, diabetes, history of cigarette smoking, congestive heart failure, malnutrition, obesity, and spinal cord injury.

In general, the wound is irrigated with normal saline or sterile water and debridement of necrotic tissue and callous completed. A povidone composition is then applied to the wound. The form of the povidone composition may depend on such factors as the surface area of the wound to be covered, type of wound being treated, and location of the wound. For example, a povidone composition in the form of a gel, cream, or ointment may be useful for ulcers and burns, while gauze impregnated with a povidone solution may be useful for surgical or traumatic wounds.

Other Applications

In addition to wound healing, it has also been surprisingly found that the povidone compositions have anti-infective and anti-inflammatory properties, and thus may be used to treat infection and/or inflammation affecting the skin. Further, the povidone compositions have also surprisingly been found to provide analgesia. Thus, the povidone compositions may be used as analgesics so that when applied, pain is relieved in addition to wounds being healed.

Suitable medical conditions (including skin conditions) where the aforementioned properties of the povidone compositions may be helpful include, without limitation, abscess, acne (including inflamed acne), carbuncles, cellulitis, dental carries, ecthyma, eczema, erysipelas, fasciitis, felon, furuncles, gangrene, herpes infections (e.g., cold sores and herpetic whitlow), hidradenitis, impetigo, mastitis, onychomycosis, otitis externa, paronychia, psoriasis, rosacea, septic bursitis, thrush, and irritation due to allergies or chemicals. It will be understood that “skin” is meant to include any layer of the skin and mucosa.

Here the povidone compositions may be directly applied to the skin or provided on a substrate, such as a wipe, tissue, or swab, for application to the skin. The povidone composition may be used for its local anti-infective and/or anti-inflammatory effect. Thus, oral medications, e.g., oral antibiotics, may also be used in combination with the topical povidone composition to systemically treat the infection or inflammation.

### III. KITS

The kits described here may include one or more povidone compositions and instructions for use. One or more substrates may optionally be included. In some instances, an applicator for spreading the povidone compositions may also be provided. The povidone compositions included in the kits may have the same topical form or different topical forms. The same or different amounts of the povidone compositions may also be employed. Substrates may also have the same or different form. The substrates may also be of varying shape and thickness.

In some variations, the povidone compositions are provided as single-use packages. When the composition is in particulate form, the povidone powder may be dispersed or sprinkled onto the wound from a container having a plurality of small holes at its outlet, such as the type of container that is generally used for the application of talcum powder. The container may be made from a flexible or resilient material so that the container may be squeezed to help expel the powder. Other topical forms may be provided in tubes, jars, or other containers having a manual pump, atomizer, and the like. The kits may also be packaged with an energy source, such as a unit or device for application of electrical energy, acoustic energy, ultrasound, or laser energy.

### IV. EXAMPLES

### Example 1

### Povidone with Non-Healing Dog Bite Wounds

A female patient presented with six non-healing dog bite wounds, one on her forearm, and the rest on her lower extremeties. Her wounds were treated with a course of antibiotics (a cephalosporin and flagyl) and daily dressing changes. After five weeks, her wounds remained unhealed and were very painful. Further treatment with hyperbaric oxygen was being contemplated.

Approximately one tablespoon of povidone powder was sprinkled over the patient's wounds. Within four days, her wounds had completely healed.

### Example 2

### Povidone with Non-Healing Skin Excision Wound

A diabetic female with a melanoma on her back had the cancer surgically removed. The resulting excision wound measured approximately 3.0 cm×3.0 cm, and extended into the dermis. Her wound was treated with dressing changes, topical antibiotics, and oral Keflex® tablets without improvement.

Approximately one tablespoon of povidone powder was applied several times a day. Within one day, the wound began to granulate. After one week of treatment, the wound was completely healed.

### Example 3

### Povidone with Non-Healing Venous Stasis Ulcer

A diabetic male presented with a stage II venous stasis ulcer on his right lower extremity. After one month of dressing changes, the wound remained unhealed. Approximately one tablespoon of povidone powder was then applied to the wound daily. Total healing of the wound resulted after one week of povidone treatment.

1. A wound dressing comprising a povidone composition wherein the povidone composition includes a povidone polymer capable of healing a wound.

2. The wound dressing of claim 1 wherein the povidone composition consists essentially of povidone particles.

3. The wound dressing of claim 2 wherein the povidone polymer comprises crosslinked povidone or a copolymer of povidone.

4. The wound dressing of claim 1 wherein the povidone composition is formulated as a cream, film, gel, lotion, ointment, paste, solution, or spray.

5. The wound dressing of claim 1 wherein the povidone composition is provided on a substrate.

6. The wound dressing of claim 5 wherein the substrate comprises a woven, non-woven, or adhesive material.

7. The wound dressing of claim 1 further comprising a vitamin, a mineral, or an herb.

8. A method for treating wounds comprising applying a povidone composition to the surface of a wound.

9. The method of claim 8 wherein the povidone composition consists essentially of povidone particles.

10. The method of claim 9 wherein the povidone particles are applied by sprinkling them over the wound surface.

11. The method of claim 10 wherein sprinkling of the povidone particles is followed by covering the wound surface with a substrate.

12. The method of claim 8 wherein water and povidone particles are mixed to form the povidone composition.

13. The method of claim 8 wherein the povidone composition is a cream, film, gel, lotion, ointment, paste, solution, or spray.

14. The method of claim 8 wherein the povidone composition is placed on a substrate before application to the wound surface.

15. The method of claim 8 wherein the wound is a burn, a diabetic ulcer, an ischemic ulcer, a pressure sore, a surgical excision, a traumatic wound, or a venous stasis ulcer.

16. The method of claim 8 further comprising reapplying the povidone composition to the wound surface.

17. A kit for treating wounds comprising:
a) one or more povidone compositions;
b) optionally, one or more substrates; and
c) instructions for use.

18. The kit of claim 17 wherein the povidone compositions are separately packaged in single use amounts.

19. The kit of claim 17 wherein the povidone compositions are the same dosage form.

20. The kit of claim 17 wherein the povidone compositions are different dosage forms.

21. The kit of claim 17 wherein the substrates have the same form.

22. The kit of claim 17 wherein the substrates have different forms.
